(19) United States
(12) Patent Application Publication
(10) Pub. No.: US 2020/0023090 A1
(43) Pub. Date: Jan. 23, 2020

(54) System and Method for Tracking Sterilized Items and Sterilizers

(71) Applicant: Sterilog Corp., Toronto (CA)

(72) Inventors: Samuel Axelrod, Toronto (CA); Zachary Axelrod, Toronto (CA)

(73) Assignee: Sterilog Corp., Toronto (CA)

(21) Appl. No.: 16/158,764

(22) Filed: Oct. 12, 2018

(30) Foreign Application Priority Data
Jul. 20, 2018 (CA) 3012007

(51) Int. Cl. A61L 2/24; A61L 2/28

(52) U.S. Cl. CPC A61L 2/24; A61L 2/0023; A61L 2/28

(57) ABSTRACT

The disclosure relates to a device and method for tracking status of sterilization for a sterilized package being sterilized in a sterilizer. The method comprises executing instructions on a or at a computing device that: track in a database a status of a first sterilization test relating to the sterilized package being sterilized in the sterilizer; track in the database a status of a second sterilization test relating to the sterilized package being sterilized in the sterilizer; generate on a display controlled by the computing device a graphical user interface (GUI) showing the status of the first and second sterilization tests; and upon a change of the status of the first sterilization test in the database, update the GUI to show an updated status of the first biological test.

### FIELD OF THE DISCLOSURE

The present disclosure relates to systems and methods for tracking packages of sterilized items (or items prepared for such sterilization), where sterilized items include tools, implements, devices, materials, etc. Such sterilized items are used in various fields, such as dentistry, medicine, physiotherapy, veterinary, cosmetology, etc. The present disclosure also relates to systems and methods for tracking sterilizing devices that sterilize those items.

### DESCRIPTION OF THE BACKGROUND

Current sterilizing devices include stand-alone sterilizers and autoclaves. In use, items that are to be sterilized can be placed and sealed in a plastic package (bag or pouch) and then the package and its contents are placed into a sterilizer. Once a sterilization cycle is complete, the bag is removed.

Current sterilization tracking systems involve affixing handwritten labels (or directly writing on the package with pen or marker) that identify the sterilized item in a package with separate manual logs to track the package. Manual logs track labelled bags containing sterilized items.

Current sterilization processes place packages to be sterilized into a sterilizer with a chemical indicator of a sterilization cycle parameter on the outside of the package (e.g. a Type 1 indicator) with another, more demanding indicator placed inside the package (e.g. a Type 4 indicator). Multiple indicators may be placed in a package. A biological indicator (BI) test vial may also be placed in the sterilizer with the package. Proper verification of sterilization mandates that following a sterilization cycle in the sterilizer, the packages are quarantined for an incubation period for the test vial in an incubator. A load of packages placed in a sterilizer/incubator may also have a BI test or a Type 5 test indicator loaded. A Type 5 indicator indicates whether packages in that load have been exposed to certain minimum steam pressure, temperature and/or time exposures. Once the BI test vial indicates that the packages are sterile, the instruments may be noted as being sterilized and may be released for use. Incubation periods may be up to 24 hours, which and as such, an instrument being sterilized may not be available for use during a day.

Certain regulators for health professionals, such as dentists, define processes and standards for sterilization of instruments. To decrease the overall time to determine that a sterilized instrument has passed all sterilization/incubation procedures, regulations permit conducting a single biological test per day and permit the packages to be released for use before the results are known of the test, providing that another test, called a type 5 indicator is included with the packages in the sterilizer.

Staff maintain separate log books for packages being sterilized and for tracking of biological tests, but the log records are not co-ordinated. It is also difficult to track when a BI test vial just removed from the sterilizer is placed into an incubator. After completion of an incubation period (e.g. 24 hours), staff remove the vials from the incubator and review the test results. Results are manually tracked (pass/fail) for the sterilized package and any control vial with other data.

Current tracking systems lack sufficient information, coordination and timeliness of information. Sterility of instruments becomes an issue as operators handle paper documents, log books, storage drawer handles, pens etc., thereby increasing a possibility of cross-contamination.

There is a need to address deficiencies in the prior art.

### SUMMARY OF THE DISCLOSURE

In a first aspect of an embodiment for this disclosure, a method for tracking status of sterilization for a sterilized package being sterilized in a sterilizer by executing instructions on a processor at a computing device is provided. The method comprises: tracking in a database a status of a first sterilization test for a first indicator relating to the sterilized package being sterilized in the sterilizer; tracking in the database a status of a second sterilization test for a second indicator relating to the sterilized package being sterilized in the sterilizer; generating on a display controlled by the computing device a graphical user interface (GUI) showing the status of the first and second sterilization tests; and upon a change of the status of the first sterilization test in the database, updating the GUI to show an updated status of the first biological test.

The method may further comprise: extracting identification data from the database relating the sterilized package; and sending instructions to a printer connected to the computing device to print a label with the identification data.

The method may further comprise: tracking in the database a status of a first biological test in an incubator relating to the sterilized package being sterilized in the sterilizer; and generating on the display in the GUI an indicator showing the status of the first biological test mimicking a layout of vials in the incubator.

The method may further comprise: updating the database with an active status of the first biological test for a third indicator; and updating on the display in the GUI the indicator to show the active status of the first biological test.

In the method, the third indicator may be a biological Indicator (BI) or a Type 5 indicator for a load being sterilized with the sterilized package.

The method may further comprise: tracking in data in the database relating to a certification time for the first biological test; when the certification time has elapsed analyzing in data for completion of certification of the first biological test; and if the certification time has elapsed and completion of certification has not been completed, generating in the GUI a visual indicator to indicate non-completion of the certification.

The method may further comprise tracking reprocessing data for the sterilized package in the database.

In a second aspect, a computing device for tracking status of sterilization for a sterilized package being sterilized in a sterilizer is provided. The computing device comprises: a memory storage device; a communication link to an electronic database; and a processor. The processor executes instructions that: track in the database a status of a first sterilization test for a first indicator relating to the sterilized package being sterilized in the sterilizer; track in the database a status of a second sterilization test for a second indicator relating to the sterilized package being sterilized in the sterilizer; generate on a display controlled by the computing device a GUI showing the status of the first and second sterilization tests; and upon a change of the status of the first sterilization test in the database, update the GUI to show an updated status of the first biological test.

In the computing device, the processor may execute further instructions that: extract identification data from the database relating the sterilized package; and send instructions to a printer connected to the computing device to print a label with the identification data.

In the computing device, the processor may execute further instructions that: track in the database a status of a first biological test for a third indicator in an incubator relating to the sterilized package being sterilized in the sterilizer; and generate on the display in the GUI an indicator showing the status of the first biological test mimicking a layout of vials in the incubator.

The third indicator may be a BI or a Type 5 indicator for a load being sterilized with the sterilized package.

In the computing device, the processor may execute further instructions that: update the database with an active status of the first biological test; and update on the display in the GUI the indicator to show the active status of the first biological test.

In the computing device, the processor may execute further instructions that: track data in the database relating to a certification time for the first biological test; when the certification time has elapsed, analyze the data for completion of certification of the first biological test; and if the certification time has elapsed and completion of certification has not been completed, generate in the GUI a visual indicator to indicate non-completion of the certification.

A server and/or a device may be provided to implement any aspects of the method described.

In other aspects various combinations of sets and subsets of the above aspects are provided.

### BRIEF DESCRIPTION OF THE DRAWINGS

Embodiments of the disclosure will now be described, by way of example only, with reference to the accompanying drawings, in which:

FIG. 1A is a schematic block diagram of an electronic package tracking system for sterilizers with a printer, connected to a network, which is connected to a server and a database according to an embodiment;

FIG. 1B is a schematic block diagram of exemplary printed labels created by the printer as controlled by the electronic package tracking system of an embodiment as shown in FIG. 1A;

FIG. 2 is a schematic block diagram of software modules operating on the electronic package tracking system of an embodiment as shown in FIG. 1A;

FIGS. 3-12 are schematic block diagrams of separate software modules operating on the electronic package tracking system of an embodiment as shown in FIG. 2; and

FIGS. 13A-13F are schematic block diagrams of graphical user interface (GUI) outputs generated on a display of the electronic package tracking system of an embodiment as shown in FIG. 1A.

### DETAILED DESCRIPTION OF EMBODIMENTS

Exemplary details of embodiments are provided herein. The description which follows and embodiments described therein are provided by way of illustration of an example or examples of particular embodiments of principles of the present disclosure. These examples are provided for the purposes of explanation and not limitation of those principles and of the disclosure. In the description which follows, like parts are marked throughout the specification and the drawings with the same respective reference numerals.

Briefly, an embodiment provides systems, methods, devices and processes for tracking sterilized items, sterilizers, incubators and sterilization/incubation test results.

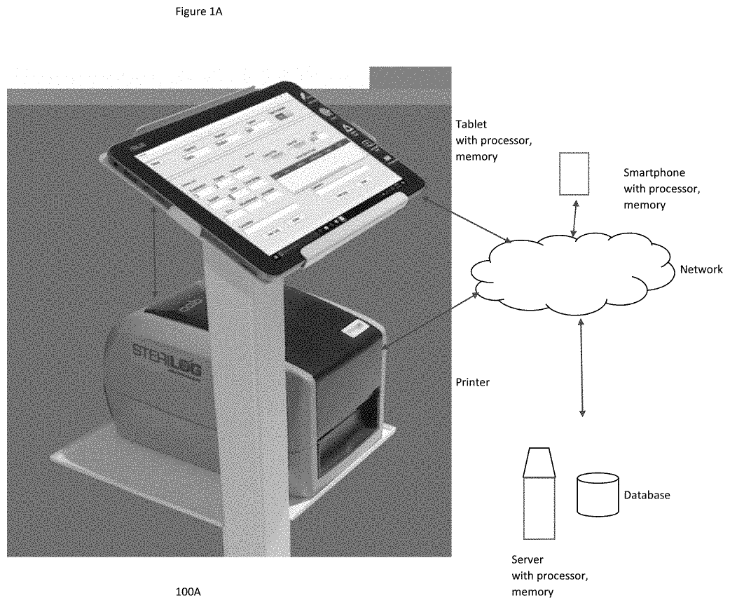

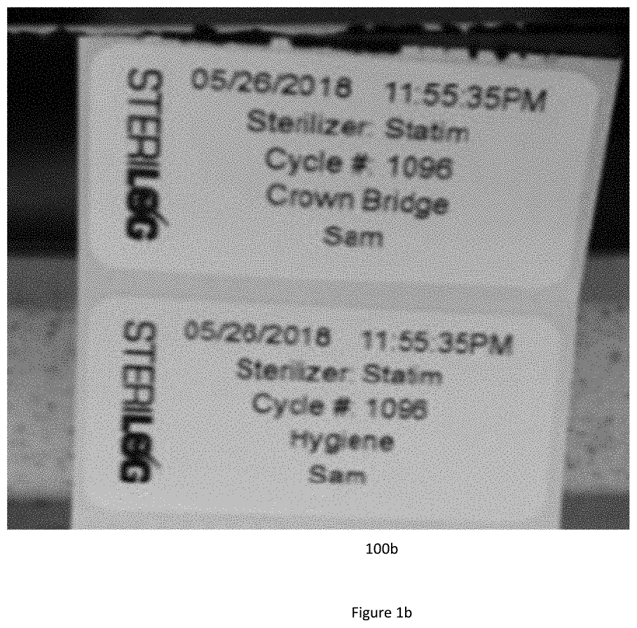

First, for context, structural components of an embodiment and its features are described in view of FIG. 1A. An embodiment of the disclosed system and method is depicted in environment 100. At its core, an embodiment has a printer that is in communication with a computing device (such as a tablet, laptop, mobile device, desktop computer, a network server, etc.) and both are in communication with a network. There is also a server and database in connection with the network and so ultimately in connection with the printer and computing device. Each computing device may communicate with each of its associated printer and the network through wired communication links (e.g. Ethernet) or wireless communication links (e.g. through Wi-Fi networks). A computing device may have installed thereon local client software, providing an application program interface (API) and a communication interface to the printer, the server and the database.

Within each exemplary computing device, there is a processor, memory, and communication access to the server and database. Software operating on the processor, accepts data as provided by a user, the printer, the server and the database and processes it, to provide output commands for labels to be printed on the printer and for updating and retrieving records from the database. The software also creates several graphical user interfaces (GUIs) and user interfaces (UIs) that are generated on a display of the computing device. The database and server process and store records associated with sterilizers and packages tracked by an embodiment. The database stores records and data relating user accounts, packages, contents of packages (e.g. a list of sterilized items), tests conducted on the packages and their status, tests to be conducted on the packages and their status and other relevant data, tests conducted on the sterilizers and their status, tests to be conducted on the sterilizers and their status and other relevant data. One or more functions of the computing device may be distributed among several devices. It will be appreciated that devices are clients in the network and that any client may have similar corresponding components and structures to comparable components described.

In other embodiments, the software may be stored on a remote server or other computing device, which may be callable for execution by the computing device.

Exemplary computing devices include touch screen tablet computer operating on Microsoft Windows (trademark) environment executing software that handles data inputs and processing of such data. Internal time clocks provide time and date information for date and time fields to enable tracing of results beyond regulatory tracking requirements.

Output in the form of labels may be spooled using a print spooler in the computing device to process and queue up print jobs. As print jobs complete, confirmations are returned to the print drivers and relayed back to the software for initialization of next steps.

Software for embodiments operating on a computing device or server may be developed in any computer language. such as Visual Basics for .NET Framework. Data in database may be stored in SQL databases such as Microsoft Access, SQL Server or MySQL. Commands to select or act on data from database may be provided in any appropriate database query language processor, such as SQL Structured Query Language. Outputs from the database may be exported in files readable by other computing devices, such as through Microsoft Excel.

The Tables below provide exemplary fields and details for records stored for user and sterilizers, and test results in a database:

| | |
|---|---|
| Field | Contents |

User Record

Name
PIN
Title
. . .

Sterilizer Record

| | |
|---|---|
| Location | 101 Main Street |
| Title | Main Sterilizer |
| Cycle Number | 1234 |
| . . . | |

Package Record

| | |
|---|---|
| Name | Surgery |
| Quantity | 3 |
| Time, date of sterilization | |
| Operator sterilizing package | Mary |
| Sterilizer used | Main Sterilizer |
| Cycle number | 1234 |
| Operator certifying chemical test results | John |
| Type 1 chemical test results | Pass |
| Type 4 chemical test results | Fail |
| Type 5 chemical test results | Pass |
| Physical Parameter checked results | Verified |
| Time, date of certification | |
| Notes | Free notes |
| . . . | |

Biological Indicator Record

| | |
|---|---|
| Sterilizer name | Main Sterilizer |
| Cycle number | 1234 |
| Lot number of vials | 5678 |
| Time, date of sterilization | |
| Location in Incubator | P1a |
| Time, date inserted into incubator | |
| Operator at sterilization | Mary |
| Operator at incubation | Bill |
| Operator at certification | John |
| Time, date of certification of test results | |
| Control Vial Test Results | +growth |
| Test Vial Test Results | −growth |
| Note Fields | Free notes |
| . . . | |

it will be appreciated that contents of these data records are stored and accessed by processes in an embodiment to track and display results relating to sterilizers, sterilizer packages, and test results.

Now further detail is provided on software operating on a computing device for an embodiment that prints labels for sterilized packages, tracks sterilized packages, sterilizers and users and updates a database to track aspects of the sterilized packages, sterilizers and users.

Briefly, an embodiment provides systems, methods, processes and techniques to track packages and chemical and/or biological tests.

A feature of an embodiment assists in establishing and confirming that sterilized packages are, in fact, sterile. An embodiment facilitates tracking of multiple sterilization/incubation tests for multiple sterilizers and sterilization packages, analyzes data logs relating to same. The status of various tests is analyzed and presented in a GUI in an enriched manner, providing additional information about the sterilized package, its contents, and status of various tests (including, for example, location of a vial in an incubator, date and time of entry, and operators at different stages during sterilization/incubation.

An embodiment also provides facilities to track active BI tests and present in a GUI results of related biological test grids indicating, locations of active BI tests vials (e.g. in a sterilizer or an incubator).

As well, results and tracking of sterilized packages with their chemical indicator test results and the biological test results may be stored in an accessible network location and sorted by sterilizer cycle number. This data may be combined with data from the sterilizers (e.g. time, temperature, pressure etc.) for each minute of that sterilization cycle.

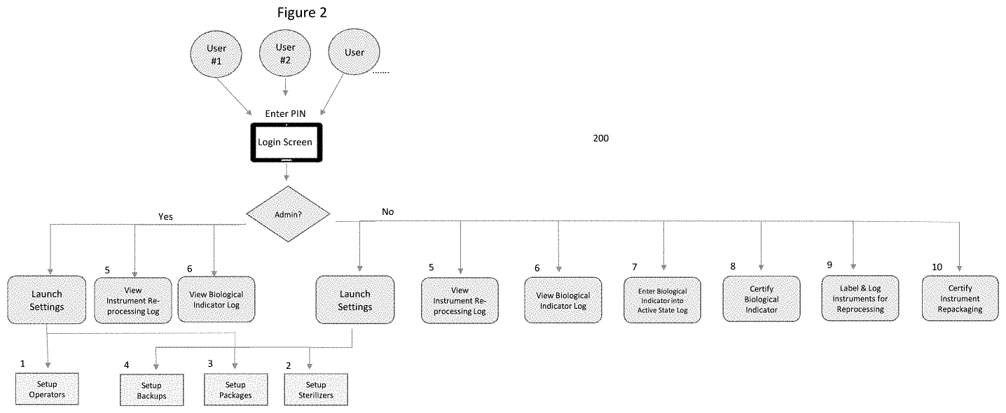

FIG. 2 provides flow chart 200 showing processes executed by software of an embodiment operating on a computing device, where users log in to the software and through multiple nested Graphical User Interfaces (GUIs) is provided with data (from the database) and options to print labels for sterilized packages, enter information into the database and retrieve information regarding sterilized packages, sterilizers and users from the database.

In FIG. 2, processes 1, 2, 3, and 4 relate to setting and modifying user setting for the software. An embodiment for the software provides four customizations of main GUI shown in the “desktop”. These setting enable customizations of operator names, user PINs, settings for backup locations, names of sterile package contents, and names of sterilizer.

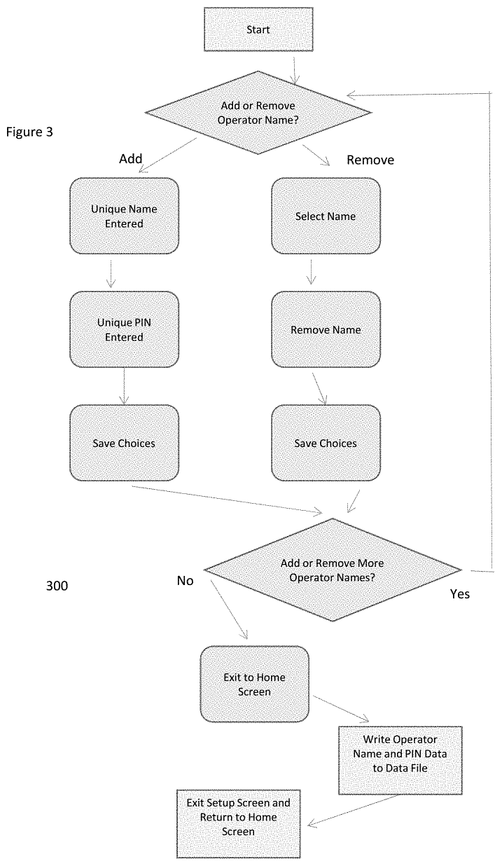

FIG. 3 illustrates process 300 showing functions executed by software for an embodiment in administering operator names (e.g. adding, removing, modifying, etc. records of operator names). Therein, software and database of an embodiment store lists of current operators for display and modification. To modify, remove or add a user, process 300 responds to an Administrative Operator key in of a unique user name in a first input field1 of a GUI generated on a display and an associated Personal Identification Number (PIN) in a second field in the GUI and selection of “Add User” button. Process 300 verifies uniqueness of contents of both fields against existing names and PINs in database. If unique, user and associated PIN is added to “operators” database and lists of current operators is refreshed and pulled from the database. An embodiment maintains a searchable record of all data recording operators for every transaction.

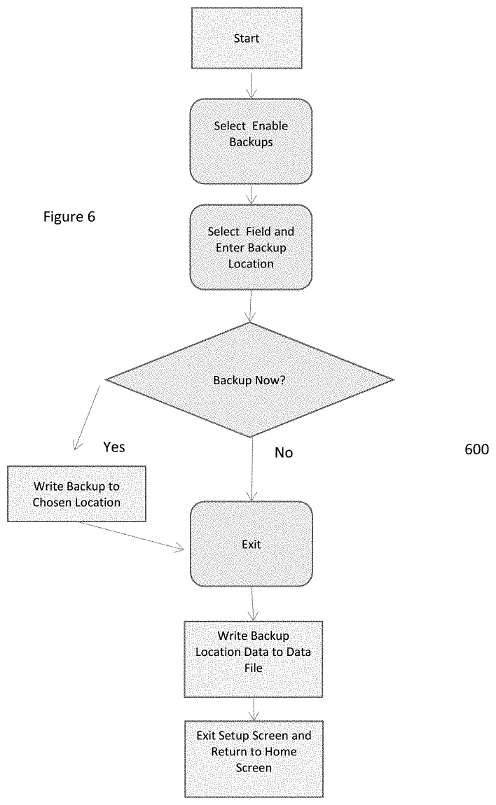

In FIG. 2, process 4 relates to setting backup locations, to which further details are provided in FIG. 6. In FIG. 6, process 600 utilizes timers to initiate backups on a prescribed frequency. A GUI provides a menu-driven to allow a user to select whether to enable/disable backups. Before triggering a timed backup, process 600 verifies a status of a switch. Process 600 reacts to a user's selection of a backup location (via a Windows File dialogue pop-up) when the user activates a “Select Location” button. Location may be stored in settings memory of the program. Upon activation of a “Backup Now” button, process 600 copies database to the backup location, independent of the timed auto-backups.

A process in an embodiment allows tracking of levels of sterilization for pouches and instruments and tracking of specific sterilization tests.

Regulations for health professionals, such as dentists, have been established by the respective health professional governing bodies as well as federal, provincial and US state health departments. Regulations relate to standards and methods for sterilizing, storing, and tracking instruments used for clinical use, be either disposable, one-use and how they are cleaned (e.g. through physical cleaning by an instrument washing system, ultrasonic cleaner or hand washing and scrubbing) and how they are stored (e.g. whether they are wrapped in a sealed pouch or wrapping material), and how they are transported.

A sealed package or wrapped group of instruments may then sterilized in an appropriate sterilizer (usually pressured steam based). This package may then be sterilized in a cycle of the sterilizer that is appropriate for the instruments in that package.

As part of a (cleaning) sterilization cycle, various chemical indicator test devices (typically test strips) may be applied to the package (either inside or outside the package) being sterilized. Results of the test devices typically provide a visual indicator of a positive or negative (cleaning) sterilizer process variables, e.g. presence of steam, temperature reached, pressures established, durations of the former, etc. and various combinations of these results.

There are several classes of chemical and biological indicators available, some of which are briefly described below.

Type I (“Class I”) indicators respond to one or more process variables. One example of a Type I chemical indicator is indicator tape that is applied to the outside of a package and primarily used to secure wrapped cassettes of instruments. When the pouch is removed from the sterilizer, the external chemical indicator will have a change in colour when it was exposed to steam and processed. An internal chemical indicator responds to a change in one or more pre-defined process variables with a chemical or physical change. An internal chemical indicator should be placed in a pouch undergoing sterilization and its placement should be in an area least accessible to steam penetration. Type IV chemical indicator strips are multi-variable indicators and react to two or more critical variables in the sterilization cycle as specified by the manufacturer. A biological Indicator (BI) spore test may require approximately between 1 and 24 hours of quarantined instruments after processing the BI test, depending on the spore test being used.

A Type 5 indicator is placed in each sterilizer load in the sterilizer and indicates whether the packages in that load have met required steam pressure, temperature and time exposures. Regulations allow operators to release packages for use that are awaiting BI results if a load includes a passed Type 5 indicator.

Ultrasonic clean test strips are foil covered with a looser material that comes apart when the ultrasonic cleaner has provided enough kinetic energy to dislodge the material, and hence particulate matter on instruments. Aluminum foil testing is a strip of foil placed into the ultrasonic cleaner to test effectiveness and strength of the ultrasonic vibrations. If sufficient, numerous holes will appear in the foil. Bowie-Dick testing is a chemical test that determines the amount of pre-cycle vacuum developed in System B sterilizers. A process in an embodiment tracks date of testing, operator, sterilizer, cycle number, and results for these tests to assist with correlation results to each other and to the instrument package contents.

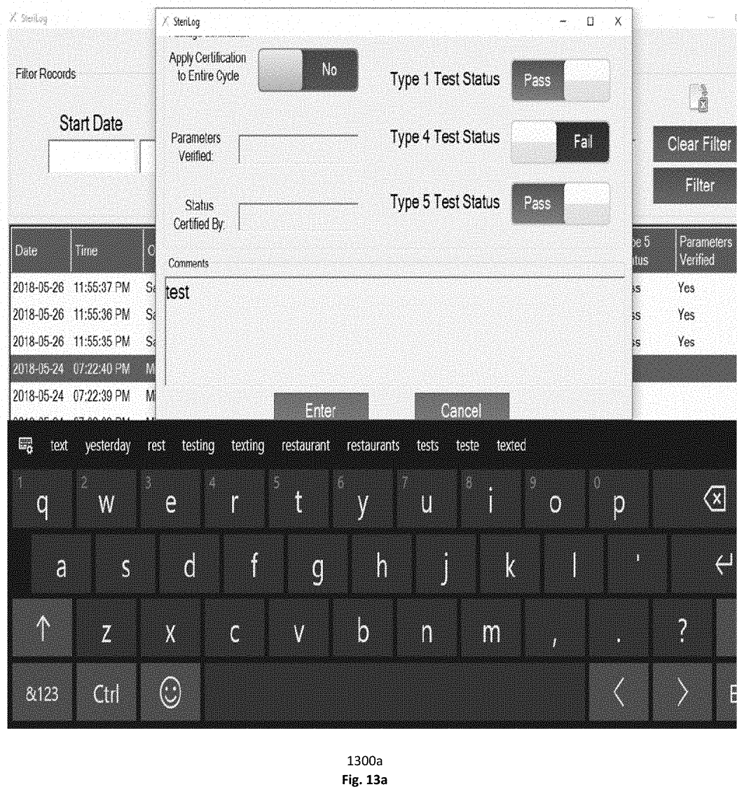

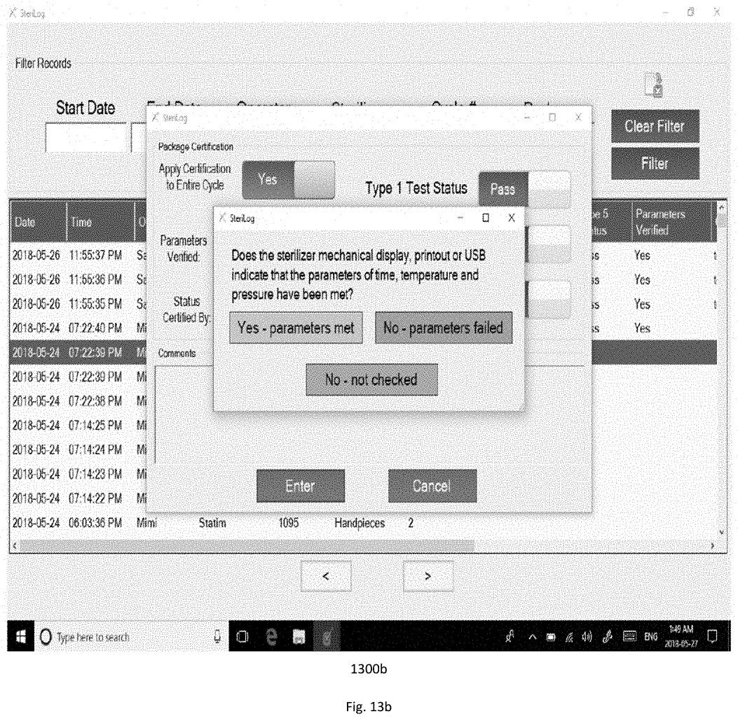

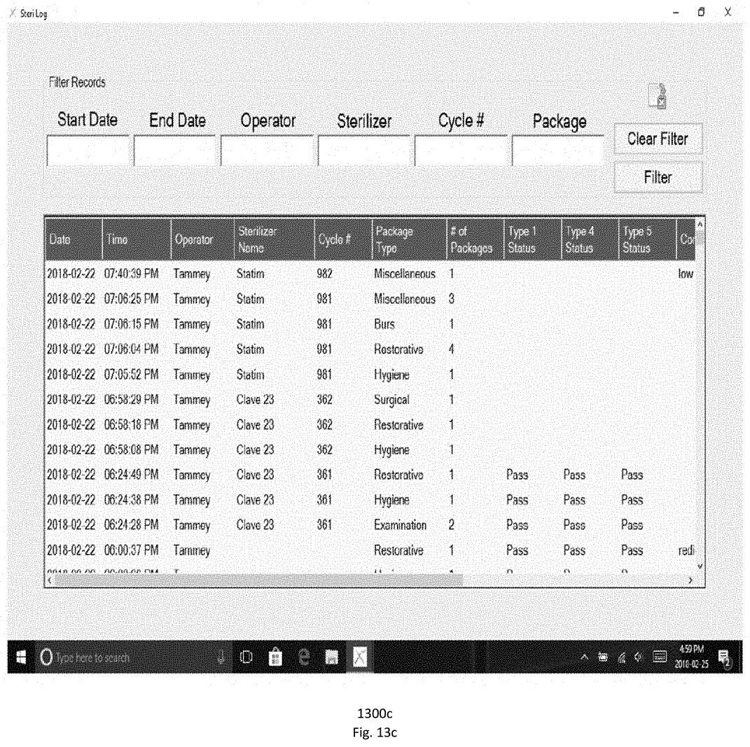

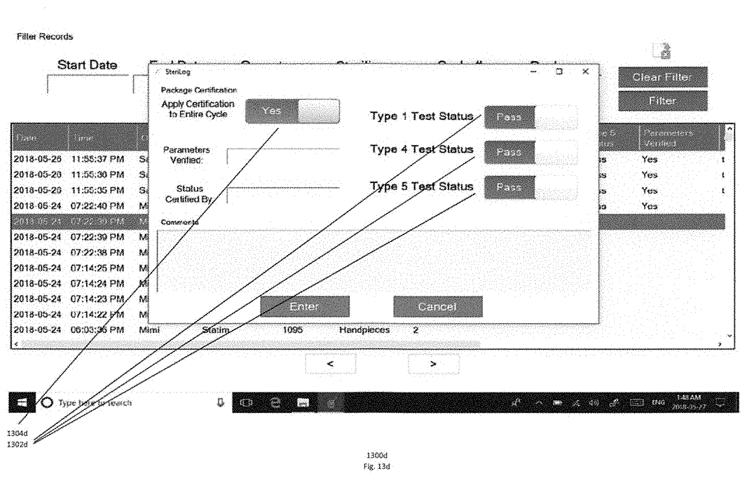

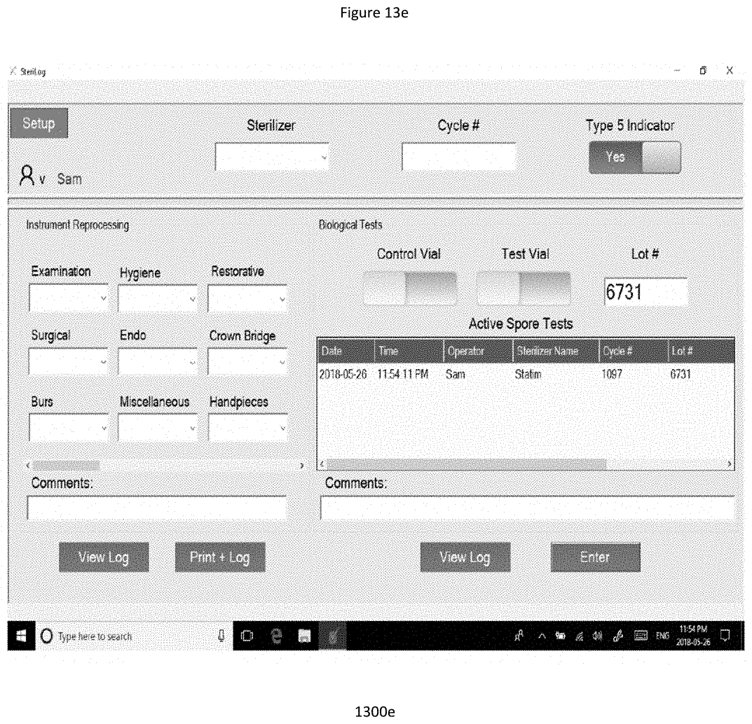

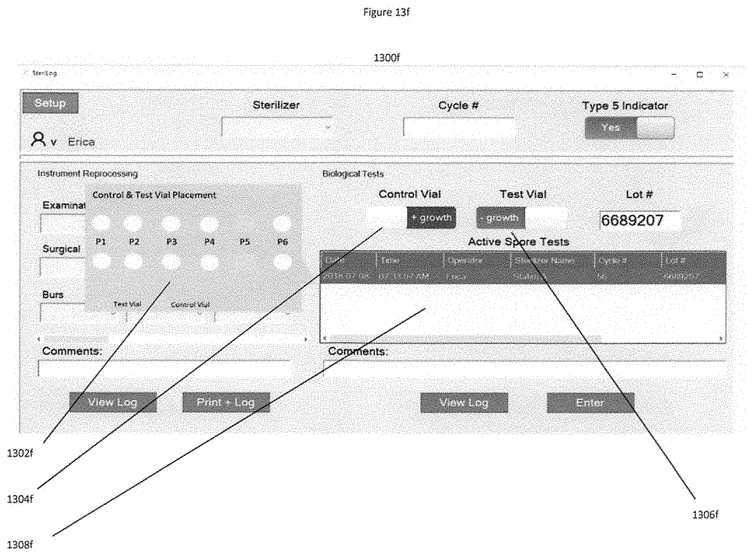

Results of sterilization/incubations for the indicators for the sterilized packages and/or loads are tracked by an embodiments. The embodiment also generates accessible and visually enhanced results of the indicators in a GUI. See for example, in FIG. 13F GUI 1300F, provides GUI indicator buttons showing information on control of vial placements, indicators for control vials and test vials and a log for active spore tests in one location.

An embodiment provides facilities that may encourage proper recording of tracking data for a sterilized package. For a sterilized package, when its database record has a sterilization test added to it, a time/date stamp is associated with that test. An embodiment provides a trigger based on the time/date stamp that results for the test are expected to be entered into the record within a certain period of time (e.g. three days to accounts for weekends, etc.). If an operator does not certify the results by the end of that time, an embodiment will generate a prompt in a GUI to enter the result.

Occasionally, an instrument requires to be quarantined. If it is not possible to quarantine the instrument until the BI result is known, then an internal Type V chemical indicator may be used with the instrument or the associated sterilizer load during sterilization. If the indicator with the instrument shows a passed Type V or VI chemical indicator result and any applicable physical parameter(s) for quarantine for the instrument (e.g. time, temperature, pressure, etc.) have been satisfied, this net set of conditions for the instrument clear the instrument for use. Internal chemical indicators are checked at the point of use, prior to using the instrument. An embodiment facilitates tracking of the various test indicators and parameters for an instrument and data analysis on same can be used to flag when a quarantined device is able to be used.

Also, in the database, reprocessing logs and BI logs contain data reflecting whether or not certain tests have been completed. An embodiment analyzes the data and generates visual indicators of these items in a GUI in a dashboard BI active log portion of the GUI, indicating whether or not that these items are not yet completed.

As a pouch completes sterilization, results for the test device may be examined by an operator. A process in an embodiment provides facilities to track successful or non-successful completion of these indicator tests with data for the pouches. As well, an embodiment provides numerous notations for the package on a printable label for the pouch, such as the name of operator, sterilizer used, sterilizer cycle number, contents of the package, and date of sterilization. This data is tracked in a database, which facilitates adherence to tracking regulations.

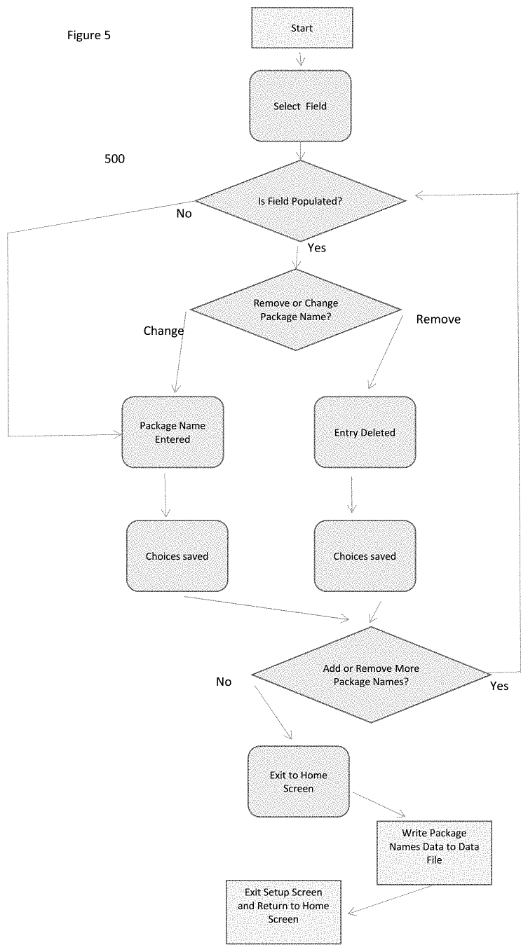

In FIG. 2, process 3 relates to viewing data relating to sterile package names, to which further details are provided in FIG. 5. In process 500, default package names are stored in a database of an embodiment. These names may be loaded into a main dashboard list of package names available for sterilization or “instrument reprocessing”.

Lists of current package names may be loaded into a data view from database for display on the computing device. In adding package names, process 500 responds to entry of a key-in of package names into an input field of a GUI followed by activation of an “Add Package” button in the GUI. Package names may be added to “Package names” database and lists of current package names may be refreshed and pulled from the database.

To modify existing names, process 500 responds to a selection of a particular package, the keying in of changes and the selection of the “Save Changes” button. Process 500 creates and utilizes references particular package names via indexing and SQL select commands and updates existing package name with the changes. Changes are reflected and loaded onto main dashboard for display.

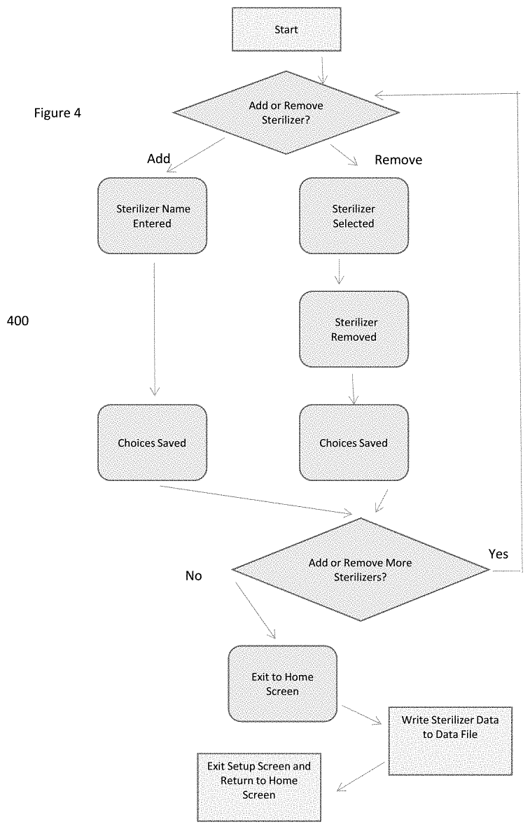

In FIG. 2, process 2 relates to setting up sterilizer names, to which further details are provided in FIG. 4. In process 400, a sterilizer name may be called up on a main GUI dashboard dropdown function list of sterilizer names available. Lists of current sterilizer names may be loaded into a data view from database for display. To add sterilizer names, process 400 responds to a key in of a new sterilizer name into an input field of a GUI and activation of an “Add Sterilizer” button. Sterilizer names are added to a sterilizer names database and lists of current sterilizer names is refreshed and pulled from the database. A process in an embodiment responds to the selection of a particular sterilizer, the keying in of changes and the selection of “Save Changes” button to modify existing names. A process in an embodiment may access the database and extract an active cycle value, increment the cycle value in sequence and then display that number in a GUI.

System references particular sterilizer names via indexing and SQL select commands and updates existing sterilizer name with the changes. Changes may be reflected and loaded onto main dashboard GUI sterilizer combination box.

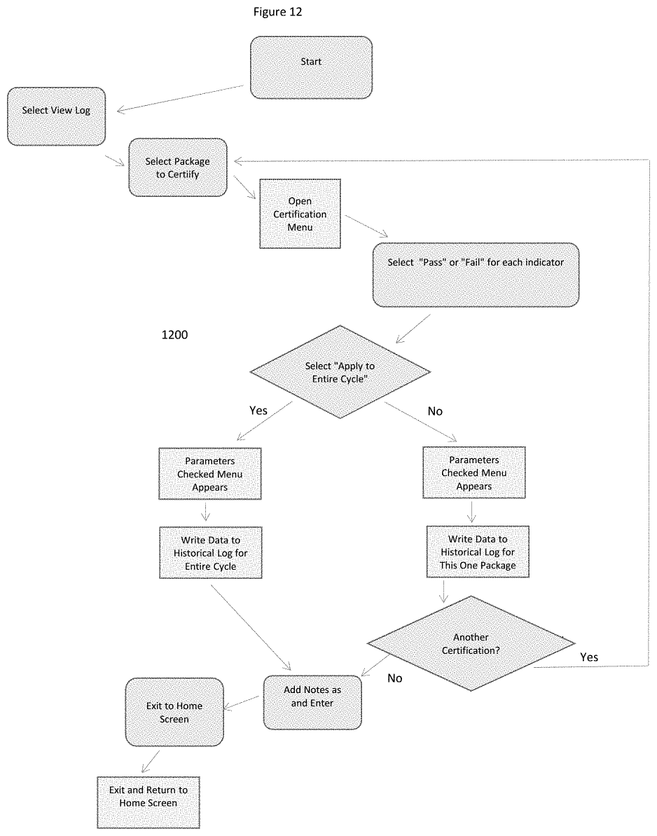

In FIG. 2, process 5 relates to viewing data relating to instrument reprocessing, to which further details are provided in FIG. 12. In process 1200, once a package is sterilized, a log may be re-accessed to record success or non-success (“pass/fail”) of the chemical tests for that particular package. All these steps require signature or initial of the person performing each process. An embodiment updates relevant GUIs to reflect the status as shown in FIG. 13a with GUI 1300a and in FIG. 13b with GUI 1300b showing indicators of verification of physical sterilizer function parameters.

From a dashboard GUI, an operator may select “View Instrument Reprocessing Log” action. A Windows form may be launched containing a data view that is populated with log data by binding data view to database in background system process. The fields displayed may include: package name, cycle number, sterilizer name, time stamp, date stamp, chemical indicator test result, operator name and fields for notes, which may record user comments, error corrections, etc.

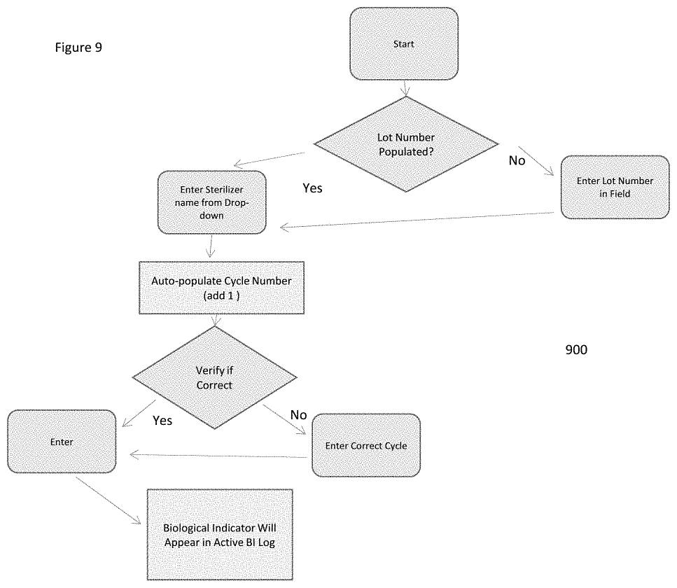

In FIG. 2, process 6 relates to viewing BI logs. In FIG. 9, process 900 generates logs for BI tests for separate data sources and data log display. When a test vial is activated and placed into a sterilizer, by regulation, the operator must log this event and its parameters. After the vial completes the sterilization cycle, it is removed and placed in an incubator for a manufacturer-specific temperature and time period. The position of the vial in the incubator is selected/marked by an embodiment as represented in FIG. 13f in GUI 1300f. After a required time elapses, the operator reads vial colours from the indicators and logs success or non-success of both the test and the control vial. At this point, the vials are discarded and no further recording is required.

Recording positions of vials in the incubator enables an operator to visually identify which vial is from which sterilizer and when its incubation cycle began (if at all). Once the data record is selected pertaining to a vial, GUI will show a graphical representation of various test results from indicators expected for that vial. Once the vial is certified as to the test results, an embodiment updates the status of the vial, resets location data for the vial, updates the related visual indicated in the GUI and transfers the relevant data to the BI log.

An embodiment maintains and tracks the data pertaining to that particular test, including parameters of test results, in a separate log format in the database for record-keeping and filtering based on various parameters.

It will be appreciated that there may be confusion as to whether or not an active BI test is in progress, either in a sterilizer or in an incubator receptacle. By providing and maintaining a visual log display of active BI tests on a dashboard in a GUI, an embodiment enables an operator, at a glance, to recognize not only that active BI vials are present, but the locations in the incubator of such vials, once placed there.

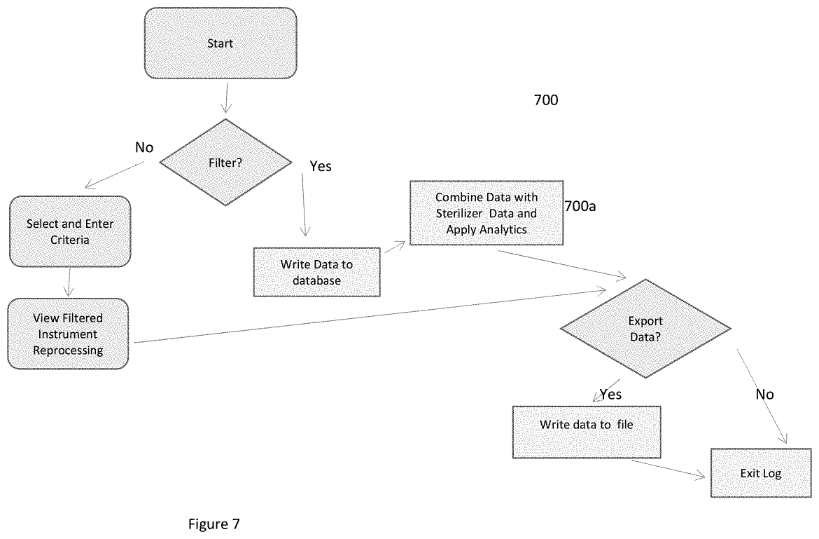

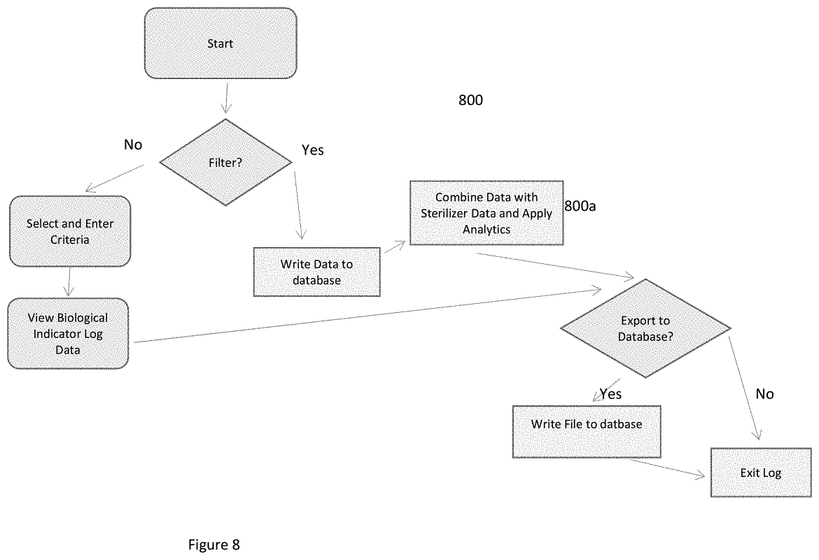

FIGS. 7 and 8 show processes for viewing logs for reprocessed instruments and BI tests respectively; they have similar process flows and executions, including processes 700a and 800a (“Combine data with Sterilizer Data and apply Analytics”). It will be appreciated that such analytics may be developed and deployed to address specific analytical requirements for test results. Exemplary analytics may be any of: analyzing the number of cycles were executed in a day against historic data; variances in cycles from day to day data; cycle histories for particular package types compared against other package types. Such analytics may assist in managing instrument inventory and office staff workflows.

FIG. 13f shows elements of GUI 1300f generated by an embodiment that shows various graphical status indicators of multiple sterilization/incubation tests. Generation of display of pop-up incubator grid 1302f graphically represents a top view of an actual vial incubator, mimicking grid locations on an actual incubator with three vials in position. For example, an Ensure (trademark) incubator may have vials in its P1a and P2a locations, showing test vials for two separate sterilizer cycles. The incubator may have in P1b, for a third “Control” vial. Correspondingly, data for the incubator in an embodiment tracks vials P1a, P2a and P1b and generates grid 1302f showing a representative incubator grid that enables an operator to mark a location of a particular vial representing its actual location in the actual incubator. By clicking the spot in grid 1302f, the location status changes on the GUI and the database is updated accordingly. This coordination facilitates an operator certifying a test vial to check the GUI to locate the correct vial in the actual incubator, thereby avoiding vial identification errors.

In an embodiment, when a grid location is selected as having a particular “vial” in it, the database is updated to block that grid location from being assigned to another vial until the current vial is certified and then deleted from the GUI. This assists in preventing additional errors.

As well, the cell number of the incubator slot represented in the GUI and a real incubator (such as P1a, P2a, and P1b in a real incubator as noted above) is recorded in the BI database and an embodiment accesses this data to generate on a GUI in a data view for reference per grid 1308f.

It will be appreciated that when an operator examines an entry in the data view where an “Active Biological Indicator Log” does not have an incubator position displayed, the operator may assume that the vial is likely still in a sterilizer. This is because an embodiment registers a vial in the “Active BI Log” only upon its entering to the sterilizer. Once the vial is removed and placed in the incubator and the status is changed for the vial, an embodiment then updates the graphical status of the vial in the GUI and updates an “Active BI Log” to reflect the position in the incubator.

When a vial is certified by an operator as to “+growth” or “−growth”, a record of that vial is deleted from the “Active BI Log” and an embodiment updates the GUI to reflect same (per indicators 1304f and 1306f). All steps in the process are date, time stamped as well as operator involved. Per process 1000, when displaying the main dashboard GUI, process 1000 responds to a selection of sterilizer name and then conducts searches to extract current cycle number from the database. Process 1000 displays in a GUI previously entered lot number for the vial from records extracted from the database and outputs it to text field on GUI dashboard. The embodiment will change the lot number by responding to a selection of the field, and change in this number. A change in colour is generated to represent whether an actual vial is “infected” or “not infected”. A colour may be changed from grey to default red (+growth) when the data field is selected, representing whether the vial is “infected” or not, showing an actual state for the control vial. If a vial is properly sterilized and all the parameters of the sterilizer cycle has been met, the vial should appear without a colour change indicating that it is sterile. In an embodiment, when the operator selects “green”, representing (−growth) on a toggle slide button in GUI 1300f, the related status for that vial is updated accordingly in the database. Selections for the status slide between green and red.

The embodiment will update the record to replace the previous number in the database. When the operator selects an “Enter Biological Indicator” button is selected, process 1000 verifies privileges of the operator and then, if appropriate, writes the operator name, sterilizer name, cycle number, time and date stamps and comments to a storage location for active biological test data. Active biological indicator data on main dashboard GUI may be refreshed by rebinding and refreshing data drawn from the database.

When a BI is moved from a sterilizer to an incubator, the operator would select the Biological Test from the BI Active GUI view, to which process 100 generates a display of a grid form. A process in an embodiment responds to selection of a location on this grid for placement of both the test and control vials representing the actual placement of such vials in the physical incubator. Data relating to sterilizer location, operator name, date, time, etc. may be written to a biological indicator log in an embodiment.

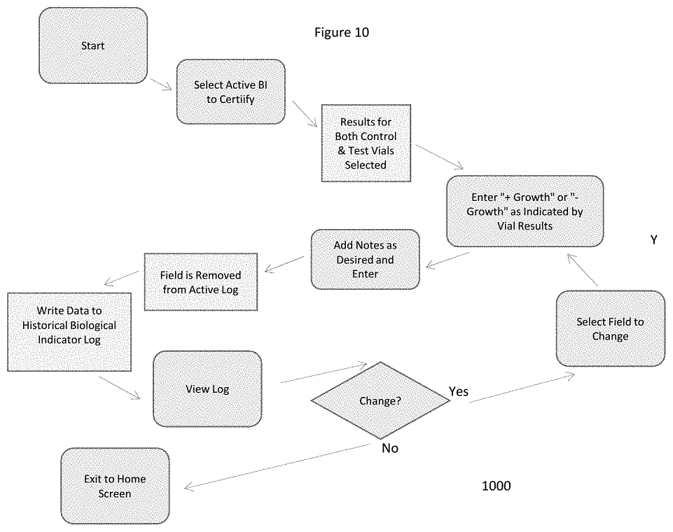

In FIG. 2, process 8 relates to certifying BI results. In FIG. 10, process 1000 implements same. Regulated health records may require keeping an audit trail of who produced that record and when. A process in an embodiment promotes authenticity of such records by extracting the certifiers name from the log-in. Once recorded in the database, preferably this cannot be altered, except via a note to file which ensures a traceable audit trail.

In process 1000, a GUI generates information on an Active BI data record. Process 1000 responds to an operator's selection of a particular record for certification. Process 1000 then highlights the selection and enables and displays two switches for indication of +growth/−growth for the control and test vials. These are highlighted in distinctive colours. Toggling of these two switches to reflect state of the test vials and the selection of “Enter” will set the state of the test vials in the system. Process 1000 writes the data to a BI log database and adds secondary operator name, time, date, test results, control results and additional comments to that log database. Process 1000 then locates and removes this record from a transient BI database via a SQL delete query.

An active BI data record on a GUI dashboard may be refreshed by rebinding and drawing data from the transient database reflecting removal of the record. A pop-up data grid GUI that previously showed active vials in the incubator is modified to reflect the removal of these vials and those fields previously populated will revert to the default “empty” indication.

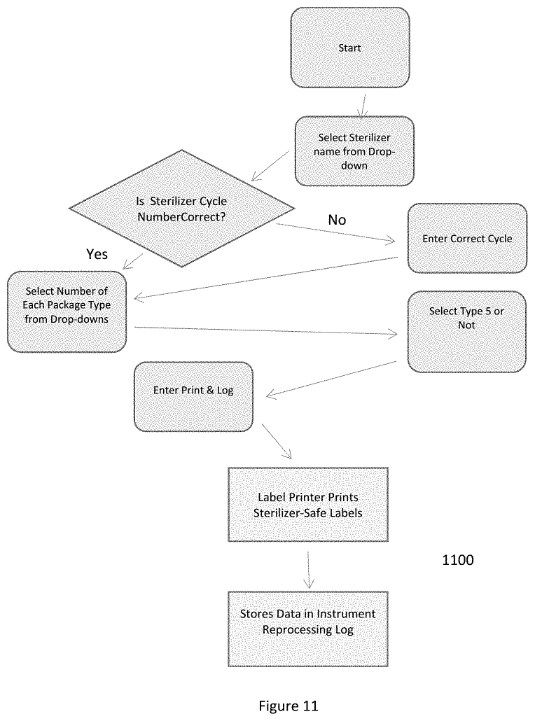

In FIG. 2, process 9 relates to labeling and logging instruments for reprocessing, to which further details are provided in FIG. 11 in process 1100. Therein, from a main dashboard GUI, after selection of a particular sterilizer name, an embodiment recalls a last cycle number from a record in the database and generates a next sequential cycle number and writes it to the cycle text field in the record. This number may be modified if the field be selected and the existing number overwritten. Process 1100 replaces the previous number in the record in the database. Selection of a switch on the dashboard will indicate to the system whether a “Type 5” test is being used in this sterilizer load.

As noted earlier, Type 5 tests are required under certain regulations to allow release of instruments from a particular sterilizer load if a B1 indicator had not completed within a full incubation period. A Type 5 test strip may be placed into every active load unless BI indicators are used and the instrument packages are held in quarantine pending the incubation period termination and the ability to determine the results.

User preferences are stored in memory and toggle switch state may be set to default matching this preference. Selection of a number of packages occurs for a particular available package name via a combination box dropdown menu corresponding to the number of each Instrument Package type to be physically loaded into the sterilizer. Process 1100 responds to the selection of the “Enter” button on the dashboard. Process 1100 verifies privileges of the current operator. If validation criteria are met, process 1100 loops through lists of available package names and number of packages from populated package type fields and populates a parameterized template for printing labels with the associated date, time, operator, sterilizer name, package type and operator name associated with the package.

The template may be sent to the print spooler of the pre-configured thermal transfer printer and printed with the number of copies corresponding to the selected number of packages selected from the package name combination box. Process 1100 loop continues for all selected package fields until exhausted. See FIG. 1B for an exemplary printer output shown at 100b.

Labels 100b have information as to the time of sterilization, operator and may include an additional bar code containing additional encoded information.

Data associated with each package is written to the “instrument reprocessing log” in the database.

In FIG. 2, process 8 relates to certifying biological/chemical indicators, to which further details are provided in FIG. 12 in process 1200. An embodiment reduces operator error and provides complete records for all packages by prompting an operator for any un-certified packages appearing in the log after pre-selected time period (e.g. 24, 48, or 72 hours). An embodiment provides a visual indicators 1302d in GUI 1300d (FIG. 13d), providing visual indicators confirming or not confirming that all instrument reprocessing packages have had their test result recorded. As well, an embodiment permits an operator to “apply result to entire cycle” (button 1304d) to allow the operator to certify the test results all at once, usually if successful. A process in an embodiment also allows the operator to selectively return to the record of any package to change the test result record, should a single, or multiple packages fail the testing.

A process in an embodiment uploads this data to the server to merge of it with data from the sterilizer itself for each cycle. This provides a unified record of every sterilizer cycle that includes information for time, temperature and pressure at each timed interval of the sterilization cycle, instrument reprocessing data from the log data view.

A process in an embodiment permits visually highlighting in a GUI a data record for a particular package in a cycle in response to selection of that item in an Instrument Reprocessing log. Therein, an embodiment may call up a Windows display form with toggle switches corresponding to various chemical indicator test results (e.g. “Type1”, “Type 4”, “Type 5”, etc.). A comment box is displayed as well. Another GUI box provides the name of the certifying operator on subsequent displays of this form. These toggles are adjusted by the operator to reflect the state of these chemical tests on the actual package/load and the results of the toggles are updated in the database. An additional toggle will indicate to the system to apply these results to the entire cycle. Upon activation of “enter” button, these results are registered and a secondary Windows Form is displayed prompting operator to select one of three possible scenarios as to having checked the physical sterilizer mechanical function. A process in an embodiment runs a series of queries to update the database based on these and previous selections of the operator. Changes associated with certifications of these packages are stored in a separate audit trail database. A process in an embodiment may rebind the instrument reprocessing log data view with its associated database and refreshes displayed data reflected updates made.

A process in an embodiment also provides remote server data synchronization. Sterilizer data is stored in database file format and uploaded to the remote server. Data previously uploaded from sterilizers may be combined in a server based database via a SQL data query on sterilizer name, cycle number and date. The combined data indicating sterilizer, cycle number, time, temperature, pressure of sterilizer, load contents, operator, chemical indicator certification may be stored in a new database. The database may be remotely accessible by an operator for printing on another printer or for downloading data to a local computing device.

A process in an embodiment also provides tracking of several users with different identification codes and access privileges. A system log-in GUI is generated (FIG. 2) at initiation of program, operator selected log-out via log-out button on main GUI, or after a predetermined time of inactivity. To dismiss the log-in screen and register a user, an embodiment responds to entering a PIN and selection of “enter”. The embodiment verifies the PIN against the “operators” database and if a match is found, the Log-in form is closed and a main GUI dashboard is displayed with functions limited to the permissions of the current user. Otherwise, a log-in failure message is generated.

As a safety feature, when a process of an embodiment detects selection of any text or comment field, the process will first verify keyboard engagement is enabled in settings. If so, the process will verify whether or not TabTip (a Windows on screen keyboard) or a comparable onscreen keyboard is currently active in the window.

If not currently displayed, the process will launch activation of the keyboard. Should the system detect contact in any other non-text field on the screen, the program will send a system command to close the onscreen keyboard window.

A process in an embodiment also provides recording sterilizer cycle data from visual inspection of manual sterilizers. For this process, in data log the operator records the temperature and pressure of the sterilizer from observation of manual gauges on non-recording sterilizers.

A process in an embodiment also provides scanning of bar-coded (or like encoded) instrument reprocessing package labels into patient charts for use in health professional office management software, permitting an operator to scan labeled packages into an office management software, establishing that a particular package was used for that patient appointment. This provides compliance with is a regulatory requirement and provides an audit trail of each instrument package from reprocessing, to indicator testing to end-user patient.

Another feature of an embodiment provides remote automated reading of results from sterilizers having mechanical gauges indicating various statuses. A remote image capturing device (such as a camera) may be located in front of the gauges and the device may periodically capture images of the reading on the gauges. An embodiment processes the images to extract data relating to the reading and such data may automatically be added to the database tracking the sterilizer, its mechanical parameters, data for time-stamped pressure and temperature readings, and packages contained therein.

Based on features of an embodiment as described above, it will be appreciated that an embodiment provides at least one or more of the following features (in any combination):
Tracking of various chemical tests for packages being sterilized and previously sterilized. Such tests include Type 1, Type 4, Type 5, Bowie Dick, Ultrasonic clean test, Aluminum Foil test, BI results, etc. in one location. This assists with a reviewer to locate all required chemical and biological test results in a log that is keyed into the sterilizer cycle number for each sterilizer as well as the package contents;
Indicators for multiple chemical tests are collected and displayed in a common GUI. This assists an operator to quickly see whether there are test vials in an incubator and sterilizers, without having to physically open the sterilizer to locate the vials or inspect the actual incubator for same. The actual locations in a grid format for the vials in the incubator slots exist as a pop-up display grid in the application. This allows the operator to avoid confusion as to which vial in the incubator corresponds to which sterilizer test. In addition, an embodiment records the name of the operator and the time that the vial was transferred from a sterilizer to an incubator;
Unique PIN based login ensures security and traceability on data entered. An embodiment facilitates a proper audit trail this by providing a secure PIN based login that reverts back to the null screen after a minute of non-use;
Audit trail of all processes. The audit trail ensures that all data changes are recorded automatically, which assists in error detection. This audit trail assists with compliance with related sterilization tracking regulations;
Diagnostics on combined data. The remote database combines uploaded data from multiple computing devices into a single data group for each cycle of the sterilizer;
Certification of multiple packages in a sterilizer load simultaneously. An embodiment facilitates certification of multiple packages in a particular sterilizer load by selecting “apply to entire load” certification option;
Filtering of data logs;
Autoclave safe labeling;
Non-alterable certification of test results and tracking of operator entries for improved reliability and tracking of data entries;
Flexible printing of labels and data logs. All logs and labels may be time-stamped to facilitate traceability; and
Flexible data entries—notes can be added to all data entries.

The various features described above may be implemented in, and fully automated by processes executed by general-purpose computing devices, including but not limited to data center servers, PCs, tablets, laptops and mobile phones. The processes may be stored in any type or types of computer storage device or memory. It should be understood that the various steps may alternatively be implemented in-whole or in-part within specially designed hardware.

It will be appreciated that all processes, servers, managers, agents, and modules described herein for computing device, database, client applications on computing devices and other sessions, processes, steps or functions in embodiments may be implemented using known programming techniques, languages and algorithms, such as Java, C++, and others. Although the processes, services and modules described are implemented in applications on computing devices, it will be appreciated that some functions of the processes may be provided in a separate server that is in communication with computing devices or its servers. The titles of processes and platforms are provided as a convenience to provide labels and assign functions to certain processes. It is not required that a process perform only its functions as described above. As such, specific functionalities for each application or process may be moved between processes or separated into different processes. Processes may be contained within other processes. Different signaling techniques may be used to communicate information between applications using known programming techniques. Known data storage, access and update algorithms allow data to be shared between applications. It will further be appreciated that other applications and systems on devices and servers may be executing concurrently with other processes. As such, any of modules (or parts thereof) may be structured to operate in as a “background” application on devices and servers, respectively, using programming techniques known in the art.

It will be appreciated that the embodiments relating to clients, servers, services, state machines and systems may be implemented in a combination of electronic hardware, firmware and software. The firmware and software may be implemented as a series of processes, applications and/or modules that provide the functionalities described herein. The algorithms and processes described herein may be executed in different order(s). Interrupt routines may be used. Data may be stored in volatile and non-volatile devices described herein and may be updated by the hardware, firmware and/or software.

As used herein, the wording “and/or” is intended to represent an inclusive-or. That is, “X and/or Y” is intended to mean X or Y or both.

In this disclosure, where a threshold or measured value is provided as an approximate value (for example, when the threshold is qualified with the word “about”), a range of values will be understood to be valid for that value. For example, for a threshold stated as an approximate value, a range of about 25% larger and 25% smaller than the stated value may be used. Thresholds, values, measurements and dimensions of features are illustrative of embodiments and are not limiting unless noted. Further, as an example, a “sufficient” match with a given threshold may be a value that is within the provided threshold, having regard to the approximate value applicable to the threshold and the understood range of values (over and under) that may be applied for that threshold.

Although this disclosure has been described in terms of certain embodiments and applications, other embodiments and applications that are apparent to those of ordinary skill in the art, including embodiments which do not provide all of the features and advantages set forth herein, are also within the scope of this disclosure. Accordingly, the scope of the present disclosure is intended to be defined only by reference to the following claims.

1. A method for tracking status of sterilization for a sterilized package being sterilized in a sterilizer by executing instructions on a processor at a computing device, the method comprising:
tracking in a database a status of a first sterilization test from a first indicator relating to the sterilized package being sterilized in the sterilizer;
tracking in the database a status of a second sterilization test a second indicator relating to the sterilized package being sterilized in the sterilizer;
generating on a display controlled by the computing device a graphical user interface (GUI) showing the status of the first and second sterilization tests; and
upon a change of the status of the first sterilization test in the database, updating the GUI to show an updated status of the first biological test.

2. The method for tracking status of sterilization for a sterilized package being sterilized in a sterilizer as claimed in claim 1, further comprising:
extracting identification data from the database relating the sterilized package; and
sending instructions to a printer connected to the computing device to print a label with the identification data.

3. The method for tracking status of sterilization for a sterilized package being sterilized in a sterilizer as claimed in claim 2, further comprising:
tracking in the database a status of a first biological test for a third indicator in an incubator relating to the sterilized package being sterilized in the sterilizer; and
generating on the display in the GUI an indicator showing the status of the first biological test mimicking a layout of vials in the incubator.

4. The method for tracking status of sterilization for a sterilized package being sterilized in a sterilizer as claimed in claim 3, wherein:
the third indicator is a biological Indicator (BI) or a Type 5 indicator for a load being sterilized with the sterilized package.

5. The method for tracking status of sterilization for a sterilized package being sterilized in a sterilizer as claimed in claim 3, further comprising:
updating the database with an active status of the first biological test; and
updating on the display in the GUI the indicator to show the active status of the first biological test.

6. The method for tracking status of sterilization for a sterilized package being sterilized in a sterilizer as claimed in claim 4, further comprising:
tracking in data in the database relating to a certification time for the first biological test;
when the certification time has elapsed analyzing in data for completion of certification of the first biological test; and
if the certification time has elapsed and completion of certification has not been completed, generating in the GUI a visual indicator to indicate non-completion of the certification.

7. The method for tracking status of sterilization for a sterilized package being sterilized in a sterilizer as claimed in claim 5, further comprising:
tracking reprocessing data for the sterilized package in the database.

8. A computing device for tracking status of sterilization for a sterilized package being sterilized in a sterilizer, the computing device comprising:
a memory storage device;
a communication link to an electronic database; and
a processor executing instructions that:
track in the database a status of a first sterilization test for a first indicator relating to the sterilized package being sterilized in the sterilizer;
track in the database a status of a second sterilization test for a second indicator relating to the sterilized package being sterilized in the sterilizer;
generate on a display controlled by the computing device a graphical user interface (GUI) showing the status of the first and second sterilization tests; and
upon a change of the status of the first sterilization test in the database, update the GUI to show an updated status of the first biological test.

9. The computing device as claimed in claim 8, wherein the processor executes further instructions that:
extract identification data from the database relating the sterilized package; and
send instructions to a printer connected to the computing device to print a label with the identification data.

10. The computing device as claimed in claim 9, wherein the processor executes further instructions that:
track in the database a status of a first biological test for a third indicator in an incubator relating to the sterilized package being sterilized in the sterilizer; and
generate on the display in the GUI an indicator showing the status of the first biological test mimicking a layout of vials in the incubator.

11. The computing device as claimed in claim 10, wherein the processor executes further instructions that:
update the database with an active status of the first biological test; and
update on the display in the GUI the indicator to show the active status of the first biological test.

12. The computing device as claimed in claim 10, wherein:
the third indicator is a biological Indicator (BI) or a Type 5 indicator for a load being sterilized with the sterilized package.

13. The computing device as claimed in claim 10, wherein the processor executes further instructions that:
track data in the database relating to a certification time for the first biological test;
when the certification time has elapsed, analyze the data for completion of certification of the first biological test; and
if the certification time has elapsed and completion of certification has not been completed, generate in the GUI a visual indicator to indicate non-completion of the certification.

14. The computing device as claimed in claim 13, wherein the processor executes further instructions that:
track reprocessing data for the sterilized package in the database.
